(19) United States
(12) Patent Application Publication
(10) Pub. No.: US 2026/0286354 A1
(43) Pub. Date: Sep. 24, 2026

(54) RNA-Based Compositions and Methods for Treating Breast Cancer

(71) Applicant: Avicenna Biotech Research, LLC, Potomac, MD (US)

(72) Inventors: Babak Behnam, Clarksburg, MD (US); Hassan Fazilaty, Zurich (CH)

(73) Assignee: Avicenna Biotech Research, LLC, Potomac, MD (US)

(21) Appl. No.: 19/472,760

(22) PCT Filed: Apr. 11, 2024
(86) PCT No.: PCT/US2024/023961

§ 371 Date: Oct. 6, 2025

Related U.S. Application Data
(60) Provisional application No. 63/496,052, filed on Apr. 14, 2023.

(51) Int. Cl. C12N 15/113

(52) U.S. Cl. CPC C12N 15/113; C12N 15/1137; C12Y 207/11022; C12Y 304/24024; C12N 2310/14

(57) ABSTRACT

This invention provides an RNA molecule that (i) specifically binds to SOX11 mRNA, SNAI1 mRNA, SNAI2 mRNA, ZEB1 mRNA, CCNB1 mRNA, CCNB2 mRNA, CCNA2 mRNA, CDK1 mRNA, SPANXA1 mRNA, SPANXB1 mRNA, POSTN mRNA, or MMP2 mRNA, and (ii) has a 3′ residue that is either U or A. This invention also provides related siRNA molecules, therapeutic compositions, and methods of using same to treat breast cancer.

This application claims the benefit of U.S. Provisional Application No. 63/496,052, filed Apr. 14, 2023, the contents of which are incorporated herein by reference.

Throughout this application, various publications are cited. The disclosure of these publications is hereby incorporated by reference into this application to describe more fully the state of the art to which this invention pertains.

### BACKGROUND OF THE INVENTION

### Breast Cancer

Breast cancer is the is the most frequent and the second leading cause of cancer-related death among women (Siegel, et al., 2020). The majority of deaths are due to the spread of tumor cells to secondary organs, a process called metastasis. This complex phenomenon is a result of many deregulated cellular and molecular events that lead to dramatic transformation of cells, acquiring ectopic behaviors and resistance to the immune system and therapy (Hanahan and Weinberg, 2011). Among key aspects of this intricate phenomenon are the appearance of stem-like cancer cells that support tumor renewal and expansion, as well as acquired cell invasion-migration capacity that is the driving force of the cancer's spread throughout body (Fazilaty et al., 2013; Lambert et al., 2017). Despite academic and technological advancements, metastatic breast cancer is not yet curable and that is in large part due to the complex nature of this disease.

Cellular heterogeneity and plasticity are two essential aspects that contribute to the complexity of metastatic process and therefore need to be targeted for an efficient treatment. The existence of many different cell types makes treatment strategies difficult, as different cell populations show a variety of responses to each drug and as a result, even combinational therapies fail. Cellular plasticity exerts another layer of complexity by substituting cell populations through transformational changes. For instance, certain non-stem cancer cells can acquire stem capacities, and even by targeting cancer stem cells (CSCs) a hidden reservoir can take part in the relapse of this population. This behavior has been reported in physiological conditions to support an adult stem cell pool (Gehart and Clevers, 2019). In addition, targeting a type of cellular plasticity called epithelial to mesenchymal transition (EMT) has been proposed to have inverse effects on metastatic colonization. EMT is an essential developmental program that when ectopically reactivated in epithelial tissues, serves as the driving force of tumor dissemination (Nieto et al., 2016). When undergoing EMT, epithelial tumor cells acquire migratory and invasive properties that help them to delaminate and invade the surrounding tissue and intravasate through blood vessels into circulation. The twist arises when these tumor cells arrive at the distant organ in which they need to revert back to an epithelial state in order to adhere and proliferate. This results in a full-blown metastatic tumor. The latter is called mesenchymal-to-epithelial transition or MET (Nieto, 2013). Therefore, repressing EMT in patients who already have disseminated tumor cells in circulation or secondary organs may exacerbate this reverse process, i.e., by assisting metastatic colonization.

siRNA

siRNA molecules are a newly emerging type of therapeutic agent. They act by targeting the mRNA of a gene whose expression plays a causative or supportive role in the disorder being treated. A few such drugs are now commercially available.

However, for a given disease-related mRNA, it is not presently possible to predict with any certaintly which of many possible candidate siRNA molecules targeting that mRNA will actually exhibit effective RNAi activity. Instead, each candidate siRNA molecule must first be made and individually tested in cell culture before its RNAi activity, if any, is known. (U.S. Pat. No. 8,735,567)

### SUMMARY OF THE INVENTION

This invention provides an RNA molecule that (i) specifically binds to SOX11 mRNA, SNAI1 mRNA, SNAI2 mRNA, ZEB1 mRNA, CCNB1 mRNA, CCNB2 mRNA, CCNA2 mRNA, CDK1 mRNA, SPANXA1 mRNA, SPANXB1 mRNA, POSTN mRNA, or MMP2 mRNA, and (ii) has a 3′ residue that is either U or A.

This invention also provides an siRNA molecule comprising (i) the present RNA molecule as a sense strand, and (ii) an antisense RNA strand non-covalently bound thereto.

This invention provides a composition (siRNA cocktail) comprising a plurality of the present siRNA molecules, wherein (i) the composition comprises a plurality of siRNA molecule populations, and (ii) each of the siRNA molecule populations is directed to a different target mRNA than the other populations.

This invention provides a pharmaceutical composition comprising the present siRNA molecule and a pharmaceutically acceptable carrier. This invention also provides a pharmaceutical composition comprising the present siRNA cocktail and a pharmaceutically acceptable carrier.

This invention provides a method for treating a subject afflicted with breast cancer comprising administering to the subject a therapeutically effective amount of the present pharmaceutical composition.

The invention also provides a nanoparticle comprising the present siRNA molecule, a pharmaceutically acceptable carrier, and a targeting ligand.

Finally, this invention provides a method for killing a breast cancer cell comprising contacting the cell with an effective amount of the present pharmaceutical composition.

### DETAILED DESCRIPTION OF THE INVENTION

### Definitions

In this application, certain terms are used which shall have the meanings set forth as follows.

As used herein, “administer”, with respect to an agent, means to deliver the agent to a subject's body via any known method. Specific modes of administration include, without limitation, intravenous, intramuscular, oral, intratumoral, sublingual, transdermal, subcutaneous, intraperitoneal, and intrathecal administration. In one embodiment, “administer” means to deliver the agent to a subject's body via intravenous administration. In another embodiment, “administer” means to deliver the agent to a subject's body via intratumoral administration. In another embodiment, “administer” means to deliver the agent to a subject's body via subcutaneous administration.

In addition, in this invention, the present RNAs (particularly siRNA molecules) can be formulated using one or more routinely used pharmaceutically acceptable carriers appropriate for RNA (e.g., siRNA) agents. Such carriers are well known to those skilled in the art. For example, injectable drug delivery systems include solutions, suspensions, gels, microspheres and polymeric injectables, and can comprise excipients such as solubility-altering agents (e.g., ethanol, propylene glycol and sucrose) and polymers (e.g., polycaprylactones and PLGA's). Implantable systems include rods and discs, and can contain excipients such as PLGA and polycaprylactone. Also included in injectable drug delivery systems are adjuvants (e.g., analgesic adjuvants); inorganic compounds (e.g., alum, aluminum hydroxide, aluminum phosphate, and calcium phosphate); mineral oil (e.g., paraffin oil); bacterial products (killed bacteria Bordetella pertussis, Mycobacterium bovis, toxoids; nonbacterial organics (e.g., squalene); plant saponins from Quillaja, soybean, polygala senega; cytokines (e.g., IL-1, IL-2, and IL-12); Freund's complete adjuvant; and Freund's incomplete adjuvant) and buffers (e.g., phosphate buffered saline) or other diluents such as sterile water. In one embodiment, the present RNAs are formulated using sterile water, optionally with sodium hydroxide and/or phosphoric acid at a pH of around 7.0. The present RNAs can also be formulated using nanoparticle-conjugation and lipid vesicles.

As used herein, “breast cancer” includes, without limitation, luminal A breast cancer, luminal B breast cancer, HER2-positive breast cancer, and triple negative breast cancer (also known as basal-like breast cancer). In one embodiment, the breast cancer is luminal A breast cancer. In another embodiment, the breast cancer is luminal B breast cancer. In another embodiment, the breast cancer is HER2-positive breast cancer. In another embodiment, the breast cancer is triple negative breast cancer. In a further embodiment, the breast cancer is metastatic breast cancer. In yet a further embodiment, the breast cancer is not metastatic breast cancer.

As used herein, a “human subject” can be of any age, gender, or state of co-morbidity. In one embodiment, the subject is male, and in another, the subject is female. In another embodiment, the subject is co-morbid (e.g., afflicted with diabetes, asthma, and/or heart disease). In a further embodiment, the subject is not co-morbid. In still another embodiment, the subject is younger than 30 years old. In yet another embodiment, the subject is at least 30 years old, at least 35 years old, at least 40 years old, at least 45 years old, at least 50 years old, at least 55 years old, at least 60 years old, at least 65 years old, at least 70 years old, at least 75 years old, at least 80 years old, at least 85 years old, or at least 90 years old. In a further embodiment, the subject has stage 0 breast cancer, stage I breast cancer, stage II breast cancer, stage III breast cancer, or stage IV breast cancer. In a further embodiment, the subject has metastatic breast cancer. In a further embodiment, the subject has breast cancer that is not metastatic. In a further embodiment, the subject is being treated with chemotherapy, radiation therapy, and/or hormone therapy. For example, in one embodiment, the subject is being treated with bevacizumab (Avastin), sunitinib (Sutent), sorafenib (Nexavar), temsirolimus (Torisel), or temozolomide (Temodar). In a further embodiment, the subject is refractive to treatment with chemotherapy, radiation therapy, and/or hormone therapy.

As used herein, “isolated”, with respect to the present RNA molecule or siRNA molecule, means that the present RNA molecule or siRNA molecule, when present as a population of molecules, contains less than 50% of any other RNA molecule. For example, the present RNA molecule or siRNA molecule, when present as a population of molecules, is “isolated” if it contains less than 10%, less than 5%, less than 2%, less than 1%, less than 0.5%, less than 0.1%, less than 0.05%, less than 0.01%, or less than 0.001% of any other RNA molecule.

As used herein, “mRNA” means messenger RNA.

As used herein, “RNA” means ribonucleic acid.

As used herein, an RNA molecule “specifically binds” to a particular mRNA (e.g., SOX11 mRNA) if it: (i) binds to that particular mRNA with an affinity greater than that with which it binds to any other mRNA; or (ii) binds to the particular mRNA with an affinity of at least 500 M. Preferably, an RNA molecule specifically binds to the particular mRNA if it performs both of items (i) and (ii) above. In a preferred embodiment, the RNA molecule binds to the particular mRNA with an affinity of at least 100 μM, at least 10 μM, at least 1 μM, at least 500 nM, at least 300 nM, at least 200 nM, at least 100 nM, at least 50 nM, at least 20 nM, at least 10 nM, at least 5 nM, at least 1 nM, at least 0.5 nM, at least 0.1 nM, at least 0.05 nM, or at least 0.01 nM.

As used herein, an “siRNA molecule” is a double-stranded oligonucleotide, made of ribonucleotides, that interferes with the activity of mRNA expressed by a gene in a cell after the oligonucleotide is introduced into the cell. siRNA molecules are made by methods known to those skilled in the art. Such methods are described, for example, in U.S. Pat. Nos. 5,898,031, 6,107,094, 6,506,559, and 7,056,704.

As used herein, the term “subject” includes, without limitation, a mammal such as a human, a non-human primate, a dog, a cat, a horse, a sheep, a goat, a cow, a rabbit, a pig, a hamster, a rat, and a mouse (e.g., a murine xenograft tumor model). The present methods are envisioned for these non-human embodiments, mutatis mutandis, as they are for human subjects in this invention.

As used herein, a first siRNA molecule population is directed to a different “target mRNA” than a second siRNA molecule population if the first population specifically binds to an mRNA encoding a different protein than the protein encoded by the mRNA to which the second siRNA molecule population specifically binds. For example, an siRNA molecule population comprising RNA molecules that specifically bind to SOX11 mRNA (i.e., mRNA encoding SOX11) is directed to a different target mRNA than is an siRNA molecule population comprising RNA molecules that specifically bind to ZEB1 mRNA.

As used herein, a “therapeutically effective amount” of the present pharmaceutical composition used in the present therapeutic method is an amount sufficient to deliver to the subject a therapeutic amount of the present siRNA molecule therein. In one embodiment, a therapeutically effective amount of the pharmaceutical composition contains an amount of siRNA molecule sufficient to deliver from 0.1 mg to 500 mg of siRNA molecule to the subject per dose (e.g., (i) 1 mg, 2 mg, 3 mg, 4 mg, 5 mg, 10 mg, 20 mg, 30 mg, 40 mg, 50 mg, 60 mg, 70 mg, 80 mg, 90 mg, 100 mg, 150 mg, 200 mg, 250 mg, 300 mg, 350 mg, 400 mg, 450 mg, or 500 mg; (ii) 1 mg 5 mg, 5 mg to 20 mg, 20 mg to 50 mg, 50 mg to 100 mg, 100 mg to 200 mg, 200 mg to 300 mg, 300 mg to 400 mg, or 400 mg to 500 mg; (iii) 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 15 mg/kg, 20 mg/kg, 25 mg/kg, 30 mg/kg, 35 mg/kg, 40 mg/kg, 45 mg/kg, or 50 mg/kg; or (iv) 0.1 mg/kg to 0.5 mg/kg, 0.5 mg/kg to 1 mg/kg, 1 mg/kg to 10 mg/kg, 10 mg/kg to 20 mg/kg, 20 mg/kg to 30 mg/kg, 30 mg/kg to 40 mg/kg, or 40 mg/kg to 50 mg/kg). Moreover, this dose can be administered once, or preferably a plurality of times over the course of therapy. In the preferred embodiment, the therapeutically effective amount of siRNA molecule is administered as a single, one-time-only dose. In another embodiment, the therapeutically effective amount of siRNA molecule is administered as two or more doses over a period of days, weeks, or months (e.g., twice with the administrations separated by two weeks; once per month; once every two months; once every three months; once every four months; twice per year; or once per year). Additional dosing regimens are provided in the examples section below.

As used herein, “treating” a subject afflicted with a disorder shall include, without limitation, (i) slowing, stopping or reversing the disorder's progression (e.g., inhibiting breast cancer metastasis, slowing or reversing breast tumor growth rate, and reducing breast tumor size), (ii) slowing, stopping, or reversing the progression of the disorder's symptoms, (iii) reducing the likelihood of the disorder's recurrence, and/or (iv) reducing the likelihood that the disorder's symptoms will recur. In the preferred embodiment, treating a subject afflicted with a disorder means (i) reversing the disorder's progression, ideally to the point of eliminating the disorder (e.g., eliminating a breast tumor), and/or (ii) reversing the progression of the disorder's symptoms, ideally to the point of eliminating the symptoms, and/or (iii) reducing or eliminating the likelihood of relapse. In this invention, treating a subject afflicted with breast cancer includes, without limitation, (i) impeding tumor proliferation, (ii) impeding the renewal and tumor-initiating capacity of tumor cells, (iii) impeding EMT, (iv) blocking the expansion of a primary tumor, and (v) blocking the appearance and/or growth of a metastatic tumor. The treatment of breast cancer can be measured according to a number of clinical endpoints. These include, without limitation, overall survival time, progression-free survival time, time to progression, disease-free survival, event-free survival, time to treatment failure, duration of clinical benefit, duration of response, objective response rate, complete response, and health-related quality of life.

### Embodiments of the Invention

This application provides numerous synthetic siRNA molecules and related compositions and methods for treating breast cancer. These siRNA molecules collectively target the mRNAs of 12 genes whose expression is associated with breast cancer. Particularly advantageous is the use of siRNA cocktails including two, three, or more of these new siRNA molecules to treat this disorder.

Specifically, this invention provides an RNA molecule (preferably isolated) that (i) specifically binds to SOX11 mRNA and (ii) has a 3′ residue that is either U or A. This invention also provides an RNA molecule (preferably isolated) comprising a sequence selected from the group consisting of (i) GGAGAGAAUUCUACAUUUA UU; (ii) GGUGGAGUUAAAGUGAAAU UU; (iii) CCAGCAUUCUCUUAUUAAU UU; (iv) GGAGACACAAGCUCAUAAU UU; (v) GCCCAAGGAAUUUGAGAAA UU; (vi) GGUGGAAACUUUAAGUAAA UU; (vii) GGGCAAAUUAUUUGUAGAA UU; (viii) GCCGUUUCCAGAUUUGAAU UU; (ix) GGAGGAUGAAGUGUUUGAU UU; (x) GCCUCUACUACAGCUUCAA UU; (xi) GCCACUGGAUAGUCUUCAA UU; and (xii) GAGCUGUUAUCUUAGUUUA UU.

This invention provides an RNA molecule (preferably isolated) that (i) specifically binds to SNAI1 mRNA and (ii) has a 3′ residue that is either U or A. This invention also provides an RNA molecule (preferably isolated) comprising a sequence selected from the group consisting of (i) CAGAUGUCAAGAAGUACCAGUGCCA UU; (ii) GCCUUCAACUGCAAAUACU UU; (iii) CCGGGCAAUUUAACAAUGU UU; (iv) CCUGGGAGGAAGAUGUUUA UU; (v) GGGAGGAAGAUGUUUACAU UU; (vi) GUGACUAACUAUGCAAUAA UU; (vii) CCCACUCAGAUGUCAAGAA UU; (viii) GGGCAAUUUAACAAUGUCU UU; (ix) GCAGACUAGAGUCUGAGAU UU; and (x) CAGGACUCUAAUCCAGAGU UU.

This invention provides an RNA molecule (preferably isolated) that (i) specifically binds to SNAI2 mRNA and (ii) has a 3′ residue that is either U or A. This invention also provides an RNA molecule (preferably isolated) comprising a sequence selected from the group consisting of (i) CACAGGAGAAUGUAUUAAA UU; (ii) GACCCACACAUUACCUUGU UU; (iii) GUGCUGACCAACCAAAUAA UU; (iv) GCUAGAUUGAGAGAAUAAA UU; (v) GAAGCCAAAUGACAAAUAA UU; (vi) GACGCAAUCAAUGUUUACU UU; (vii) GCACAAACAUGAGGAAUCU UU; (viii) GGGAAAGAUUAGCUUUGAA UU; (ix) GAGGAUUCUUACAAAUUCA UU; (x) GACACACAUACAGUGAUUA UU; and (xi) CCACUGUACAAAGAAUUGU UU.

This invention provides an RNA molecule (preferably isolated) that (i) specifically binds to ZEB1 mRNA and (ii) has a 3′ residue that is either U or A. This invention also provides an RNA molecule (preferably isolated) comprising a sequence selected from the group consisting of (i) GGCGCAAUAACGUUACAAA UU; (ii) GGCAAGUGUUGGAGAAUAA UU; (iii) GCCCUAUCAAUGUGACAAA UU; (iv) GGCGGUAGAUGGUAAUGUA UU; (v) GGAGGAAGAAGUGGAAGAA UU; (vi) GAACCAGGCAAAGUAAAUA UU; (vii) GAUCCAGCCAAAUGGAAAU UU; (viii) CUCAGAACCAGUUGUAAAU UU; (ix) CUCUGAAAGAACACAUUAA UU; (x) GAGCAAGUGUCUGAAGAAA UU; and (xi) GUCUGGGUGUAAUCGUAAA UU.

This invention provides an RNA molecule (preferably isolated) that (i) specifically binds to CCNB1 mRNA and (ii) has a 3′ residue that is either U or A. This invention also provides an RNA molecule (preferably isolated) comprising a sequence selected from the group consisting of (i) GGCUAGUACAGGUUCAAAU UU; (ii) GAGCCUGUUAAAGAAGAAA UU; (iii) GUGACUGACAACACUUAUA UU; (iv) CUGGCUAGUACAGGUUCAA UU; (v) CAGGCUUUCUCUGAUGUAA UU; (vi) GCUGAUCCAAACCUUUGUA UU; (vii) GGACUGUCAAGAACAAGUA UU; (viii) GACAGAUGGAAAUGAAGAU UU; (ix) GAUCGGUUCAUGCAGAAUA UU; (x) GCACUUUCCUCCUUCUCAA UU; and (xi) CACUCUACCACAGCUGAAU UU.

This invention provides an RNA molecule (preferably isolated) that (i) specifically binds to CCNB2 mRNA and (ii) has a 3′ residue that is either U or A. This invention also provides an RNA molecule (preferably isolated) comprising a sequence selected from the group consisting of (i) GGCCAAGAAUGUGGUGAAA UU; (ii) GCAGCAAACUCCUGAAGAU UU; (iii) CCAGUGAUUUGGAGAAUAU UU; (iv) GGUGCAUUAUCAUCCUUCU UU; (v) GUUCCCAAAUCCGAGAAAU UU; (vi) GGACAUUGAUAACGAAGAU UU; (vii) GCUCUUGGCUUCCAAGUAU UU; (viii) GCACACUUUAGCCAAGUAU UU; (ix) GUGACUACGUUAAGGAUAU UU; and (x) GUCCUCUGGUCUAUCUCAU UU.

This invention provides an RNA molecule (preferably isolated) that (i) specifically binds to CCNA2 mRNA and (ii) has a 3′ residue that is either U or A. This invention also provides an RNA molecule (preferably isolated) comprising a sequence selected from the group consisting of (i) GCUGGCCUGAAUCAUUAAU UU; (ii) GCCUCAAAGUUUGAAGAAA UU; (iii) GUGGCCAAGUACAAAUUAU UU; (iv) GAGGGUGGGAAGCUUAUAU UU; (v) CCAGCUGUCAGGAUAAUAA UU; (vi) GUGGCAAGAUGAGAGUAAA UU; (vii) GCCAGUGAGUGUUAAUGAA UU; (viii) GACCCUCAAAUUCUGACAU UU; (ix) GGAUCUUCCUGUAAAUGAU UU; and (x) GCAUAUCUGAAUACAGUAU UU.

This invention provides an RNA molecule (preferably isolated) that (i) specifically binds to CDK1 mRNA and (ii) has a 3′ residue that is either U or A. This invention also provides an RNA molecule (preferably isolated) comprising a sequence selected from the group consisting of (i) CGGGAAAUUUCUCUAUUAA UU; (ii) CAGGACUAUAAGAAUACAU UU; (iii) CCUGGCUAAAGAUGAAUAU UU; (iv) GGCACUGAAUCAUCCAUAU UU; (v) GCCUUGGUCAGAGUAAUAA UU; (vi) GUCUCACUGUAACAACUAU UU; (vii) GCACCAUAUUUGCUGAACU UU; (viii) GAUGUAGCUUUCUGACAAA UU; (ix) GUUCUUCACAGAGACUUAA UU; and (x) CAGCUGUACUUCGUCUUCU UU.

This invention provides an RNA molecule (preferably isolated) that (i) specifically binds to SPANXA1 mRNA and (ii) has a 3′ residue that is either U or A. This invention also provides an RNA molecule (preferably isolated) comprising a sequence selected from the group consisting of (i) GAGGAAUUCAUGGAAAUAA UU; (ii) CUGCCACUGACAUUGAAGA UU; (iii) CCCGAGAGAACAGAAUCAA UU; (iv) CGCUACAGGAGGAACUUUA UU; (v) GUAGCAAGAAGCUACAUCU UU; (vi) CAUGGAAAUAAUGGUUGAA UU; (vii) CAGAGGAACUGCUGAAUGA UU; (viii) CUGACAUUGAAGAACCAAU UU; (ix) CCAUACUAGUGGUUCGCUA UU; and (x) CCAAUAUAUACAAUGGACA UU.

This invention provides an RNA molecule (preferably isolated) that (i) specifically binds to SPANXB1 mRNA and (ii) has a 3′ residue that is either U or A. This invention also provides an RNA molecule (preferably isolated) comprising a sequence selected from the group consisting of (i) GCCACUGCGAAGAUUCAAA UU; (ii) GGAGGAGGAAUUCAUAGAA UU; (iii) CCCGAGAGAACAGAAUCAA UU; (iv) CAUCGAAGAACCAAUAUAU UU; (v) CCUCAAACUUCGGCAAUGA UU; (vi) CAGAGGAACUGCUGAAUGA UU; (vii) CGAAGAACCAAUAUAUACA UU; (viii) CAUAGAAAUAACGACUGAA UU; (ix) CCAUACUAGUGGUUCGCUA UU; and (x) CAAGAAGCUACAUCCCUCA UU.

This invention provides an RNA molecule (preferably isolated) that (i) specifically binds to POSTN mRNA and (ii) has a 3′ residue that is either U or A. This invention also provides an RNA molecule (preferably isolated) comprising a sequence selected from the group consisting of (i) GCUGGCACCUGUGAAUAAU UU; (ii) CCUGGAGACUGGACAUUAU UU; (iii) GCCCUGGUUAUAUGAGAAU UU; (iv) GGUCCUAAUUCCUGAUUCU UU; (v) GCCAUCACAUCGGACAUAU UU; (vi) GGAUCUAGAAGACGAUUAA UU; (vii) GAGCCUUGUAUGUAUGUUA UU; (viii) CUGCUGGAAAUACUUAAUA UU; (ix) GUAGCACCUUCAAAGAAAU UU; and (x) GGCAUGAUUAUUCCUUCAA UU.

This invention provides an RNA molecule (preferably isolated) that (i) specifically binds to MMP2 mRNA and (ii) has a 3′ residue that is either U or A. This invention also provides an RNA molecule (preferably isolated) comprising a sequence selected from the group consisting of (i) GCCCUGAAGAAUCAGCAAU UU; (ii) GUGGCCAACUACAACUUCU UU; (iii) GAGGUUUGCUUCCUCUUUA UU; (iv) CCACCACCUACAACUUUGA UU; (v) GGUCAGAGCUUCCAAAGUA UU; (vi) GCACCCAUUUACACCUACA UU; (vii) GAGGCAGACAUCAUGAUCA UU; (viii) CUCACUCCUACCUGGUAAU UU; (ix) GUGCCUAUUACCUGAAGCU UU; and (x) GUGCCCAAGAAUAGAUGCU UU.

In a preferred embodiment, the present RNA molecule's two 3′ residues are selected from the group consisting of UU, AA, AU, and UA. Preferably, the present RNA molecule's two 3′ residues are UU.

In a preferred embodiment, the present RNA molecule is fewer than 25 residues in length. In one embodiment, the present RNA molecule is 25 residues in length. In another embodiment, the present RNA molecule is 24 residues in length. In another embodiment, the present RNA molecule is 23 residues in length.

In another embodiment, the present RNA molecule is 22 residues in length.

In another embodiment, the present RNA molecule is 21 residues in length.

This invention also provides an siRNA molecule (preferably isolated) comprising (i) the present RNA molecule as a sense strand, and (ii) an antisense RNA strand non-covalently bound thereto. This siRNA molecule may have two blunt ends, two sticky ends, or one blunt end and one sticky end. The overhang nucleotides of a sticky end can range from one to four or more. In one embodiment, the overhang is one nucleotide in length. In another embodiment, the overhang is two nucleotidee in length. In another embodiment, the overhang is three nucleotidee in length. In another embodiment, the overhang is four nucleotidee in length. The backbone of one or both RNA strands of the present siRNA molecule can be modified. Such modifications include the use of small molecules (e.g. sugar molecules such as N-acetylgalactosamine (GalNAc)), amino acid molecules, peptides, cholesterol, and other large molecules for conjugation onto the siRNA molecules.

The present RNA molecules and siRNA molecules can be made using any of the known methods for making single- and double-stranded RNAs. Such methods are described, for example, in U.S. Pat. Nos. 5,898,031, 6,107,094, 6,506,559, and 7,056,704.

This invention also provides a cell containing the present siRNA molecule. Such cells include, for example, breast cancer cells, rat epithelial keratinocytes, human foreskin fibroblasts, and mouse embryonic endothelial cells.

This invention further provides a nucleic acid (i.e., DNA or RNA) vector comprising a nucleotide sequence that encodes at least one strand of the present siRNA molecule.

This invention still further provides a cell containing the present vector. Such cells include, for example, breast cancer cells, rat epithelial keratinocytes, human foreskin fibroblasts, and mouse embryonic endothelial cells.

This invention provides a composition (i.e., a multi-mRNA targeting siRNA composition, also referred to as an “siRNA cocktail”) comprising a plurality of the present siRNA molecules, wherein (i) the composition comprises a plurality of siRNA molecule populations, and (ii) each of the siRNA molecule populations is directed to a different target mRNA than the other populations. In one embodiment, the composition comprises two siRNA molecule populations. In another embodiment the composition comprises three siRNA molecule populations.

This invention provides a pharmaceutical composition comprising the present siRNA molecule and a pharmaceutically acceptable carrier.

This invention also provides a pharmaceutical composition comprising the present siRNA cocktail and a pharmaceutically acceptable carrier. Pharmaceutically acceptable carriers for this cocktail include, by way of example, saline, sugars, polypeptides, polymers, lipids, creams, gels, micelle materials, and metal nanoparticles. In one embodiment, the carrier comprises at least one of the following: a glucose solution, a polycationic binding agent, a cationic lipid, a cationic micelle, a cationic polypeptide, a hydrophilic polymer grafted polymer, a non-natural cationic polymer, a cationic polyacetal, a hydrophilic polymer grafted polyacetal, a ligand functionalized cationic polymer, a ligand functionalized-hydrophilic polymer grafted polymer, and a ligand functionalized liposome. In another embodiment, the polymers comprise a biodegradable histidine-lysine polymer, a biodegradable polyester, such as poly(lactic acid) (PLA), poly(glycolic acid) (PGA), and poly(lactic-co-glycolic acid) (PLGA), a polyamidoamine (PAMAM) dendrimer, a cationic lipid (such as DOTAP), or a PEGylated PEL In still another embodiment, the carrier is a histidine-lysine copolymer that forms a nanoparticle with the siRNA molecules, wherein the diameter of the nanoparticle is about 100 nm to about 500 nm.

This invention provides a method for treating a subject afflicted with breast cancer comprising administering to the subject a therapeutically effective amount of the present pharmaceutical composition. In the preferred embodiment, the breast cancer is selected from the group consisting of luminal A breast cancer, luminal B breast cancer, HER2-positive breast cancer, and triple negative breast cancer. In a preferred embodiment, the breast cancer is metastatic breast cancer. In another embodiment, the breast cancer is not metastatic. Preferably, the subject is human.

In a preferred embodiment, this invention provides a method for treating a human subject afflicted with breast cancer (which can be metastatic breast cancer or non-metastatic breast cancer) comprising administering to the subject a therapeutically effective amount of the present pharmaceutical composition, wherein the composition includes different populations of the present siRNA molecules, each population targeting a different mRNA (i.e., administering an siRNA cocktail). In one embodiment, for example, the present pharmaceutical composition includes two populations of the present siRNA molecules, the first population targeting SOX11 (i.e., specifically binding to SOX11 mRNA), and the second population targeting ZEB1 (i.e., specifically binding to ZEB1 mRNA). For the sake of brevity, the present siRNA cocktails are abbreviated here by stating the target of each siRNA molecule separated by a slash. For example, “SOX11/ZEB1” would be the abbreviation for the present siRNA cocktail wherein two siRNA populations are found—the first targeting SOX11 mRNA and the second targeting ZEB1 mRNA.

The following two-population-containing siRNA cocktails (i.e., two-siRNA cocktails) are envisioned in this invention: (i) SOX11/SNAI1, SOX11/SNAI2, SOX11/ZEB1, SOX11/CCNB1, SOX11/CCNB2, SOX11/CCNA2, SOX11/CDK1, SOX11/SPANXA1, SOX11/SPANXB1, SOX11/POSTN, and SOX11/MMP2; (ii) SNAI1/SNAI2, SNAI1/ZEB1, SNAI1/CCNB1, SNAI1/CCNB2, SNAI1/CCNA2, SNAI1/CDK1, SNAI1/SPANXA1, SNAI1/SPANXB1, SNAI1/POSTN, and SNAI1/MMP2; (iii) SNAI2/ZEB1, SNAI2/CCNB1, SNAI2/CCNB2, SNAI2/CCNA2, SNAI2/CDK1, SNAI2/SPANXA1, SNAI2/SPANXB1, SNAI2/POSTN, and SNAI2/MMP2; (iv) ZEB1/CCNB1, ZEB1/CCNB2, ZEB1/CCNA2, ZEB1/CDK1, ZEB1/SPANXA1, ZEB1/SPANXB1, ZEB1/POSTN, and ZEB1/MMP2; (v) CCNB1/CCNB2, CCNB1/CCNA2, CCNB1/CDK1, CCNB1/SPANXA1, CCNB1/SPANXB1, CCNB1/POSTN, and CCNB1/MMP2; (vi) CCNB2/CCNA2, CCNB2/CDK1, CCNB2/SPANXA1, CCNB2/SPANXB1, CCNB2/POSTN, and CCNB2/MMP2; (vii) CCNA2/CDK1, CCNA2/SPANXA1, CCNA2/SPANXB1, CCNA2/POSTN, and CCNA2/MMP2; (viii) CDK1/SPANXA1, CDK1/SPANXB1, CDK1/POSTN, and CDK1/MMP2; (ix) SPANXA1/SPANXB1, SPANXA1/POSTN, and SPANXA1/MMP2; (x) SPANXB1/POSTN and SPANXB1/MMP2; and (xi) POSTN/MMP2.

In another embodiment, the present pharmaceutical composition includes three populations of the present siRNA molecules, the first population targeting a first mRNA, the second population targeting a second mRNA, and the third population targeting a third mRNA. These are referred to here as “three-siRNA cocktails.”

The following three-siRNA cocktail sets are envisioned in this invention.

Three-siRNA cocktail set 1: SOX11/SNAI2/SNAI1; SOX11/ZEB1/SNAI1; SOX11/CCNB1/SNAI1; SOX11/CCNB2/SNAI1; SOX11/CCNA2/SNAI1; SOX11/CDK1/SNAI1; SOX11/SPANXA1/SNAI1; SOX11/SPANXB1/SNAI1; SOX11/POSTN/SNAI1; SOX11/MMP2/SNAI1; SOX11/SNAI1/SNAI2; SOX11/ZEB1/SNAI2; SOX11/CCNB1/SNAI2; SOX11/CCNB2/SNAI2; SOX11/CCNA2/SNAI2; SOX11/CDK1/SNAI2; SOX11/SPANXA1/SNAI2; SOX11/SPANXB1/SNAI2; SOX11/POSTN/SNAI2; SOX11/MMP2/SNAI2; SOX11/SNAI1/ZEB1; SOX11/SNAI2/ZEB1; SOX11/CCNB1/ZEB1; SOX11/CCNB2/ZEB1; SOX11/CCNA2/ZEB1; SOX11/CDK1/ZEB1; SOX11/SPANXA1/ZEB1; SOX11/SPANXB1/ZEB1; SOX11/POSTN/ZEB1; SOX11/MMP2 ZEB1; SOX11/SNAI1/CCNB1; SOX11/SNAI2/CCNB1; SOX11/ZEB1/CCNB1; SOX11/CCNB2/CCNB1; SOX11/CCNA2/CCNB1; SOX11/CDK1/CCNB1; SOX11/SPANXA1/CCNB1; SOX11/SPANXB1/CCNB1; SOX11/POSTN/CCNB1; SOX11/MMP2/CCNB1; SOX11/SNAI1/CCNB2; SOX11/SNAI2/CCNB2; SOX11/ZEB1/CCNB2; SOX11/CCNB1/CCNB2; SOX11/CCNA2/CCNB2; SOX11/CDK1/CCNB2; SOX11/SPANXA1/CCNB2; SOX11/SPANXB1/CCNB2; SOX11/POSTN/CCNB2; SOX11/MMP2/CCNB2; SOX11/SNAI1/CCNA2; SOX11/SNAI2/CCNA2; SOX11/ZEB1/CCNA2; SOX11/CCNB1/CCNA2; SOX11/CCNB2/CCNA2; SOX11/CDK1/CCNA2; SOX11/SPANXA1/CCNA2; SOX11/SPANXB1/CCNA2; SOX11/POSTN/CCNA2; SOX11/MMP2/CCNA2; SOX11/SNAI1/CDK1; SOX11/SNAI2/CDK1; SOX11/ZEB1/CDK1; SOX11/CCNB1/CDK1; SOX11/CCNB2/CDK1; SOX11/CCNA2/CDK1; SOX11/SPANXA1/CDK1; SOX11/SPANXB1/CDK1; SOX11/POSTN/CDK1; SOX11/MMP2/CDK1; SOX11/SNAI1/SPANXA1; SOX11/SNAI2/SPANXA1; SOX11/ZEB1/SPANXA1; SOX11/CCNB1/SPANXA1; SOX11/CCNB2/SPANXA1; SOX11/CCNA2/SPANXA1; SOX11/CDK1/SPANXA1; SOX11/SPANXB1/SPANXA1; SOX11/POSTN/SPANXA1; SOX11/MMP2/SPANXA1; SOX11/SNAI1/SPANXB1; SOX11/SNAI2/SPANXB1; SOX11/ZEB1/SPANXB1; SOX11/CCNB1/SPANXB1; SOX11/CCNB2/SPANXB1; SOX11/CCNA2/SPANXB1; SOX11/CDK1/SPANXB1; SOX11/SPANXA1/SPANXB1; SOX11/POSTN/SPANXB1; SOX11/MMP2/SPANXB1; SOX11/SNAI1/POSTN; SOX11/SNAI2/POSTN; SOX11/ZEB1/POSTN; SOX11/CCNB1/POSTN; SOX11/CCNB2/POSTN; SOX11/CCNA2/POSTN; SOX11/CDK1/POSTN; SOX11/SPANXA1/POSTN; SOX11/SPANXB1/POSTN; SOX11/MMP2/POSTN; SOX11/SNAI1/MMP2; SOX11/SNAI2/MMP2; SOX11/ZEB1/MMP2; SOX11/CCNB1/MMP2; SOX11/CCNB2/MMP2; SOX11/CCNA2/MMP2; SOX11/CDK1/MMP2; SOX11/SPANXA1/MMP2; SOX11/SPANXB1/MMP2; and SOX11/POSTN/MMP2. Three-siRNA cocktail set 2: SNAI1/ZEB1/SNAI2; SNAI1/CCNB1/SNAI2; SNAI1/CCNB2/SNAI2; SNAI1/CCNA2/SNAI2; SNAI1/CDK1/SNAI2; SNAI1/SPANXA1/SNAI2; SNAI1/SPANXB1/SNAI2; SNAI1/POSTN/SNAI2; SNAI1/MMP2/SNAI2; SNAI1/SNAI2/ZEB1; SNAI1/CCNB1/ZEB1; SNAI1/CCNB2/ZEB1; SNAI1/CCNA2/ZEB1; SNAI1/CDK1/ZEB1; SNAI1/SPANXA1/ZEB1; SNAI1/SPANXB1/ZEB1; SNAI1/POSTN/ZEB1; SNAI1/MMP2/ZEB1; SNAI1/SNAI2/CCNB1; SNAI1/ZEB1/CCNB1; SNAI1/CCNB2/CCNB1; SNAI1/CCNA2/CCNB1; SNAI1/CDK1/CCNB1; SNAI1/SPANXA1/CCNB1; SNAI1/SPANXB1/CCNB1; SNAI1/POSTN/CCNB1; SNAI1/MMP2/CCNB1; SNAI1/SNAI2/CCNB2; SNAI1/ZEB1/CCNB2; SNAI1/CCNB1/CCNB2; SNAI1/CCNA2/CCNB2; SNAI1/CDK1/CCNB2; SNAI1/SPANXA1/CCNB2; SNAI1/SPANXB1/CCNB2; SNAI1/POSTN/CCNB2; SNAI1/MMP2/CCNB2; SNAI1/SNAI2/CCNA2; SNAI1/ZEB1/CCNA2; SNAI1/CCNB1/CCNA2; SNAI1/CCNB2/CCNA2; SNAI1/CDK1/CCNA2; SNAI1/SPANXA1/CCNA2; SNAI1/SPANXB1/CCNA2; SNAI1/POSTN/CCNA2; SNAI1/MMP2/CCNA2; SNAI1/SNAI2/CDK1; SNAI1/ZEB1/CDK1; SNAI1/CCNB1/CDK1; SNAI1/CCNB2/CDK1; SNAI1/CCNA2/CDK1; SNAI1/SPANXA1/CDK1; SNAI1/SPANXB1/CDK1; SNAI1/POSTN/CDK1; SNAI1/MMP2/CDK1; SNAI1/SNAI2/SPANXA1; SNAI1/ZEB1/SPANXA1; SNAI1/CCNB1/SPANXA1; SNAI1/CCNB2/SPANXA1; SNAI1/CCNA2/SPANXA1; SNAI1/CDK1/SPANXA1; SNAI1/SPANXB1/SPANXA1; SNAI1/POSTN/SPANXA1; SNAI1/MMP2/SPANXA1; SNAI1/SNAI2/SPANXB1; SNAI1/ZEB1/SPANXB1; SNAI1/CCNB1/SPANXB1; SNAI1/CCNB2/SPANXB1; SNAI1/CCNA2/SPANXB1; SNAI1/CDK1/SPANXB1; SNAI1/SPANXA1/SPANXB1; SNAI1/POSTN/SPANXB1; SNAI1/MMP2/SPANXB1; SNAI1/SNAI2/POSTN; SNAI1/ZEB1/POSTN; SNAI1/CCNB1/POSTN; SNAI1/CCNB2/POSTN; SNAI1/CCNA2/POSTN; SNAI1/CDK1/POSTN; SNAI1/SPANXA1/POSTN; SNAI1/SPANXB1/POSTN; SNAI1/MMP2/POSTN; SNAI1/SNAI2/MMP2; SNAI1/ZEB1/MMP2; SNAI1/CCNB1/MMP2; SNAI1/CCNB2/MMP2; SNAI1/CCNA2/MMP2; SNAI1/CDK1/MMP2; SNAI1/SPANXA1/MMP2; SNAI1/SPANXB1/MMP2; and SNAI1/POSTN/MMP2.

Three-siRNA cocktail set 3: SNAI2/CCNB1/ZEB1; SNAI2/CCNB2/ZEB1; SNAI2/CCNA2/ZEB1; SNAI2/CDK1/ZEB1; SNAI2/SPANXA1/ZEB1; SNAI2/SPANXB1/ZEB1; SNAI2/POSTN/ZEB1; SNAI2/MMP2/ZEB1; SNAI2/ZEB1/CCNB1; SNAI2/CCNB2/CCNB1; SNAI2/CCNA2/CCNB1; SNAI2/CDK1/CCNB1; SNAI2/SPANXA1/CCNB1; SNAI2/SPANXB1/CCNB1; SNAI2/POSTN/CCNB1; SNAI2/MMP2/CCNB1; SNAI2/ZEB1/CCNB2; SNAI2/CCNB1/CCNB2; SNAI2/CCNA2/CCNB2; SNAI2/CDK1/CCNB2; SNAI2/SPANXA1/CCNB2; SNAI2/SPANXB1/CCNB2; SNAI2/POSTN/CCNB2; SNAI2/MMP2/CCNB2; SNAI2/ZEB1/CCNA2; SNAI2/CCNB1/CCNA2; SNAI2/CCNB2/CCNA2; SNAI2/CDK1/CCNA2; SNAI2/SPANXA1/CCNA2; SNAI2/SPANXB1/CCNA2; SNAI2/POSTN/CCNA2; SNAI2/MMP2/CCNA2; SNAI2/ZEB1/CDK1; SNAI2/CCNB1/CDK1; SNAI2/CCNB2/CDK1; SNAI2/CCNA2/CDK1; SNAI2/SPANXA1/CDK1; SNAI2/SPANXB1/CDK1; SNAI2/POSTN/CDK1; SNAI2/MMP2/CDK1; SNAI2/ZEB1/SPANXA1; SNAI2/CCNB1/SPANXA1; SNAI2/CCNB2/SPANXA1; SNAI2/CCNA2/SPANXA1; SNAI2/CDK1/SPANXA1; SNAI2/SPANXB1/SPANXA1; SNAI2/POSTN/SPANXA1; SNAI2/MMP2/SPANXA1; SNAI2/ZEB1/SPANXB1; SNAI2/CCNB1/SPANXB1; SNAI2/CCNB2/SPANXB1; SNAI2/CCNA2/SPANXB1; SNAI2/CDK1/SPANXB1; SNAI2/SPANXA1/SPANXB1; SNAI2/POSTN/SPANXB1; SNAI2/MMP2/SPANXB1; SNAI2/ZEB1/POSTN; SNAI2/CCNB1/POSTN; SNAI2/CCNB2/POSTN; SNAI2/CCNA2/POSTN; SNAI2/CDK1/POSTN; SNAI2/SPANXA1/POSTN; SNAI2/SPANXB1/POSTN; SNAI2/MMP2/POSTN; SNAI2/ZEB1/MMP2; SNAI2/CCNB1/MMP2; SNAI2/CCNB2/MMP2; SNAI2/CCNA2/MMP2; SNAI2/CDK1/MMP2; SNAI2/SPANXA1/MMP2; SNAI2/SPANXB1/MMP2; and SNAI2/POSTN/MMP2.

Three-siRNA cocktail set 4: ZEB1/CCNB2/CCNB1; ZEB1/CCNA2/CCNB1; ZEB1/CDK1/CCNB1; ZEB1/SPANXA1/CCNB1; ZEB1/SPANXB1/CCNB1; ZEB1/POSTN/CCNB1; ZEB1/MMP2/CCNB1; ZEB1/CCNB1/CCNB2; ZEB1/CCNA2/CCNB2; ZEB1/CDK1/CCNB2; ZEB1/SPANXA1/CCNB2; ZEB1/SPANXB1/CCNB2; ZEB1/POSTN/CCNB2; ZEB1/MMP2/CCNB2; ZEB1/CCNB1/CCNA2; ZEB1/CCNB2/CCNA2; ZEB1/CDK1/CCNA2; ZEB1/SPANXA1/CCNA2; ZEB1/SPANXB1/CCNA2; ZEB1/POSTN/CCNA2; ZEB1/MMP2/CCNA2; ZEB1/CCNB1/CDK1; ZEB1/CCNB2/CDK1; ZEB1/CCNA2/CDK1; ZEB1/SPANXA1/CDK1; ZEB1/SPANXB1/CDK1; ZEB1/POSTN/CDK1; ZEB1/MMP2/CDK1; ZEB1/CCNB1/SPANXA1; ZEB1/CCNB2/SPANXA1; ZEB1/CCNA2/SPANXA1; ZEB1/CDK1/SPANXA1; ZEB1/SPANXB1/SPANXA1; ZEB1/POSTN/SPANXA1; ZEB1/MMP2/SPANXA1; ZEB1/CCNB1/SPANXB1; ZEB1/CCNB2/SPANXB1; ZEB1/CCNA2/SPANXB1; ZEB1/CDK1/SPANXB1; ZEB1/SPANXA1/SPANXB1; ZEB1/POSTN/SPANXB1; ZEB1/MMP2/SPANXB1; ZEB1/CCNB1/POSTN; ZEB1/CCNB2/POSTN; ZEB1/CCNA2/POSTN; ZEB1/CDK1/POSTN; ZEB1/SPANXA1/POSTN; ZEB1/SPANXB1/POSTN; ZEB1/MMP2/POSTN; ZEB1/CCNB1/MMP2; ZEB1/CCNB2/MMP2; ZEB1/CCNA2/MMP2; ZEB1/CDK1/MMP2; ZEB1/SPANXA1/MMP2; ZEB1/SPANXB1/MMP2; and ZEB1/POSTN/MMP2.

Three-siRNA cocktail set 5: CCNB1/CCNA2/CCNB2; CCNB1/CDK1/CCNB2; CCNB1/SPANXA1/CCNB2; CCNB1/SPANXB1/CCNB2; CCNB1/POSTN/CCNB2; CCNB1/MMP2/CCNB2; CCNB1/CCNB2/CCNA2; CCNB1/CDK1/CCNA2; CCNB1/SPANXA1/CCNA2; CCNB1/SPANXB1/CCNA2; CCNB1/POSTN/CCNA2; CCNB1/MMP2/CCNA2; CCNB1/CCNB2/CDK1; CCNB1/CCNA2/CDK1; CCNB1/SPANXA1/CDK1; CCNB1/SPANXB1/CDK1; CCNB1/POSTN/CDK1; CCNB1/MMP2/CDK1; CCNB1/CCNB2/SPANXA1; CCNB1/CCNA2/SPANXA1; CCNB1/CDK1/SPANXA1; CCNB1/SPANXB1/SPANXA1; CCNB1/POSTN/SPANXA1; CCNB1/MMP2/SPANXA1; CCNB1/CCNB2/SPANXB1; CCNB1/CCNA2/SPANXB1; CCNB1/CDK1/SPANXB1; CCNB1/SPANXA1/SPANXB1; CCNB1/POSTN/SPANXB1; CCNB1/MMP2/SPANXB1; CCNB1/CCNB2/POSTN; CCNB1/CCNA2/POSTN; CCNB1/CDK1/POSTN; CCNB1/SPANXA1/POSTN; CCNB1/SPANXB1/POSTN; CCNB1/MMP2/POSTN; CCNB1/CCNB2/MMP2; CCNB1/CCNA2/MMP2; CCNB1/CDK1/MMP2; CCNB1/SPANXA1/MMP2; CCNB1/SPANXB1/MMP2; and CCNB1/POSTN/MMP2.

Three-siRNA cocktail set 6: CCNB2/CDK1/CCNA2; CCNB2/SPANXA1/CCNA2; CCNB2/SPANXB1/CCNA2; CCNB2/POSTN/CCNA2; CCNB2/MMP2/CCNA2; CCNB2/CCNA2/CDK1; CCNB2/SPANXA1/CDK1; CCNB2/SPANXB1/CDK1; CCNB2/POSTN/CDK1; CCNB2/MMP2/CDK1; CCNB2/CCNA2/SPANXA1; CCNB2/CDK1/SPANXA1; CCNB2/SPANXB1/SPANXA1; CCNB2/POSTN/SPANXA1; CCNB2/MMP2/SPANXA1; CCNB2/CCNA2/SPANXB1; CCNB2/CDK1/SPANXB1; CCNB2/SPANXA1/SPANXB1; CCNB2/POSTN/SPANXB1; CCNB2/MMP2/SPANXB1; CCNB2/CCNA2/POSTN; CCNB2/CDK1/POSTN; CCNB2/SPANXA1/POSTN; CCNB2/SPANXB1/POSTN; CCNB2/MMP2/POSTN; CCNB2/CCNA2/MMP2; CCNB2/CDK1/MMP2; CCNB2/SPANXA1/MMP2; CCNB2/SPANXB1/MMP2; and CCNB2/POSTN/MMP2.

Three-siRNA cocktail set 7: CCNA2/SPANXA1/CDK1; CCNA2/SPANXB1/CDK1; CCNA2/POSTN/CDK1; CCNA2/MMP2/CDK1; CCNA2/CDK1/SPANXA1; CCNA2/SPANXB1/SPANXA1; CCNA2/POSTN/SPANXA1; CCNA2/MMP2/SPANXA1; CCNA2/CDK1/SPANXB1; CCNA2/SPANXA1/SPANXB1; CCNA2/POSTN/SPANXB1; CCNA2/MMP2/SPANXB1; CCNA2/CDK1/POSTN; CCNA2/SPANXA1/POSTN; CCNA2/SPANXB1/POSTN; CCNA2/MMP2/POSTN; CCNA2/CDK1/MMP2; CCNA2/SPANXA1/MMP2; CCNA2/SPANXB1/MMP2; and CCNA2/POSTN/MMP2.

Three-siRNA cocktail set 8: CDK1/SPANXB1/SPANXA1; CDK1/POSTN/SPANXA1; CDK1/MMP2/SPANXA1; CDK1/SPANXA1/SPANXB1; CDK1/POSTN/SPANXB1; CDK1/MMP2/SPANXB1; CDK1/SPANXA1/POSTN; CDK1/SPANXB1/POSTN; CDK1/MMP2/POSTN; CDK1/SPANXA1/MMP2; CDK1/SPANXB1/MMP2; and CDK1/POSTN/MMP2. Three-siRNA cocktail set 9: SPANXA1/POSTN/SPANXB1; SPANXA1/MMP2/SPANXB1; SPANXA1/SPANXB1/POSTN; SPANXA1/MMP2/POSTN; SPANXA1/SPANXB1/MMP2; and SPANXA1/POSTN/MMP2. Three-siRNA cocktail set 10: SPANXB1/MMP2/POSTN; and SPANXB1/POSTN/MMP2.

In one embodiment of the present therapeutic methods, the present siRNA molecule is administered in conjunction with one or more additional cancer therapeutics. Such additional cancer therapeutics include, without limitation, a taxane (e.g., paclitaxel and docetaxel), an anthracycline (e.g., doxorubicin and epirubicin), capecitabine, sacituzumab govitecan-hziy (Trodelvy®), tucatinib (Tukysa®), ribociclib (Kisqali®), palbociclib (Ibrance®), and eribulin (Halaven®). The following embodiments are exemplary. In one embodiment of the present therapeutic methods, the present siRNA molecule is administered in conjunction with a taxane. In another embodiment, the present siRNA molecule is administered in conjunction with paclitaxel. In a further embodiment, the present siRNA molecule is administered in conjunction with docetaxel. In a further embodiment, the present siRNA molecule is administered in conjunction with an anthracycline. In a further embodiment, the present siRNA molecule is administered in conjunction with doxorubicin. In a further embodiment, the present siRNA molecule is administered in conjunction with epirubicin. In a further embodiment, the present siRNA molecule is administered in conjunction with capecitabine. In a further embodiment, the present siRNA molecule is administered in conjunction with sacituzumab govitecan-hziy (Trodelvy®). In a further embodiment, the present siRNA molecule is administered in conjunction with tucatinib (Tukysa®). In a further embodiment, the present siRNA molecule is administered in conjunction with ribociclib (Kisqali®). In a further embodiment, the present siRNA molecule is administered in conjunction with palbociclib (Ibrance®). In yet a further embodiment, the present siRNA molecule is administered in conjunction with eribulin (Halaven®).

The invention also provides a nanoparticle comprising the present siRNA molecule, a pharmaceutically acceptable carrier, and a targeting ligand. Targeting ligands and targeted nanoparticles generally are known (see, e.g., Friedman, et al.).

Finally, this invention provides a method for killing a breast cancer cell comprising contacting the cell with an effective amount of the present siRNA molecule. In the preferred embodiment, the breast cancer cell is selected from the group consisting of a luminal A breast cancer cell, a luminal B breast cancer cell, a HER2-positive breast cancer cell, and a triple negative breast cancer cell. Preferably, the breast cancer cell is a human breast cancer cell (which can be metastatic breast cancer cell or non-metastatic breast cancer cell).

This invention will be better understood by reference to the examples which follow, but those skilled in the art will readily appreciate that the specific examples detailed are only illustrative of the invention as described more fully in the claims which follow thereafter.

### EXAMPLES

### Example 1—Dosing Regimens

### Regimen 1

The present siRNA molecule is administered intravenously to the subject at a dose of 0.3 mg/kg every three weeks as an infusion over 80 minutes. The siRNA molecule is packaged as a lipid complex injection, 10 mg/5 ml in a single-dose vial. Alternative doses for this regimen include, for example, 0.1 mg/kg, 0.2 mg/kg, 0.4 mg/kg, and 0.5 mg/kg.

### Regimen 2

The present siRNA molecule is administered subcutaneously to the subject at a dose of 2.5 mg/kg once per month. The siRNA molecule is packaged in its sodium form 190 mg/ml in a single-dose vial. Alternative doses for this regimen include, for example, 0.1 mg/kg, 0.15 mg/kg, 0.2 mg/kg, and 0.3 mg/kg. 0.35 mg/kg, 0.4 mg/kg, 0.45 mg/kg, and 0.5 mg/kg.

### Regimen 3

The present siRNA molecule is administered subcutaneously to the subject at a dose of 3 mg/kg once per month for three months, then 3 mg/kg quarterly. The siRNA molecule is packaged at 100 mg/0.5 ml in a single-dose vial, in sterile water optionally with sodium hydroxide and/or phosphoric acid at a pH of around 7.0. Alternative doses for this regimen include, for example, 1 mg/kg, 2 mg/kg, 4 mg/kg, and 5 mg/kg.

### Regimen 4

The present siRNA molecule is administered subcutaneously to the subject at a dose of 300 mg once, again in three months, and then every six months after that. The siRNA molecule is packaged as a pre-filled syringe, in sterile water optionally with sodium hydroxide and/or phosphoric acid at a pH of around 7.0. Alternative doses for this regimen include, for example, 100 mg, 150 mg, 200 mg, 250 mg, 350 mg, 400 mg, 450 mg, and 500 mg.

### REFERENCES

U.S. Pat. No. 5,898,031.
U.S. Pat. No. 6,107,094.
U.S. Pat. No. 6,506,559.
U.S. Pat. No. 7,056,704.
U.S. Pat. No. 8,735,567.
Aiello, N. M., and Kang, Y. (2019). Context-dependent EMT programs in cancer metastasis. Journal of Experimental Medicine 216, 1016-1026.
Brabletz, T., et al. (2018). EMT in cancer. Nature Reviews Cancer 18, 128-134.
Chung, W., et al. (2017). Single-cell RNA-seq enables comprehensive tumour and immune cell profiling in primary breast cancer. Nat Commun 8, 15081.
Davis, R. T., et al. (2020). Transcriptional diversity and bioenergetic shift in human breast cancer metastasis revealed by single-cell RNA sequencing. Nat Cell Biol 22, 310-320.
Deng, H., et al. (2018). Histone H3.3K27M Mobilizes Multiple Cancer/Testis (CT) Antigens in Pediatric Glioma. Mol Cancer Res 16, 623-633.
Fazilaty, H., et al. (2013). Crosstalk between breast cancer stem cells and metastatic niche: emerging molecular metastasis pathway? Tumor Biol. 34, 2019-2030.
Fazilaty H, Behnam B. The perivascular niche governs an autoregulatory network to support breast cancer metastasis. Cell Biol Int. 2014 June; 38 (6): 691-4.
Fazilaty, H., et al. (2016). SLUG and SOX9 Cooperatively Regulate Tumor Initiating Niche Factors in Breast Cancer. Cancer Microenvironment 9, 71-74.
Fazilaty, H., et al. (2019). A gene regulatory network to control EMT programs in development and disease. Nature Communications 10, 1-16.
Fischer, K. R., et al. (2015). Epithelial-to-mesenchymal transition is not required for lung metastasis but contributes to chemoresistance. Nature 527, 472-476.
Friedman, A. D., et al. (2013). The Smart Targeting of Nanoparticles. Curr Pharm Des. 19(35): 6315-29.
Gehart, H., and Clevers, H. (2019). Tales from the crypt: new insights into intestinal stem cells. Nat Rev Gastroenterol Hepatol 16, 19-34.
Guo, W., et al. (2012). Slug and Sox9 Cooperatively Determine the Mammary Stem Cell State. Cell 148, 1015-1028.
Hanahan, D., and Weinberg, R. A. (2011). Hallmarks of Cancer: The Next Generation. Cell 144, 646-674.
Karaayvaz, M., et al. (2018). Unravelling subclonal heterogeneity and aggressive disease states in TNBC through single-cell RNA-seq. Nat Commun 9, 3588.
Krebs, A. M., et al. (2017). The EMT-activator Zeb1 is a key factor for cell plasticity and promotes metastasis in pancreatic cancer. Nat Cell Biol 19, 518-529.
Lambert, A. W., et al. (2017). Emerging Biological Principles of Metastasis. Cell 168, 670-691.
Maine, E. A., et al. (2016). The cancer-testis antigens SPANX-A/C/D and CTAG2 promote breast cancer invasion. Oncotarget 7.
Malanchi, I., et al. (2012). Interactions between cancer stem cells and their niche govern metastatic colonization. Nature 481, 85-89.
Mani, S. A., et al. (2008). The Epithelial-Mesenchymal Transition Generates Cells with Properties of Stem Cells. Cell 133, 704-715.
Nguyen, Q. H., et al. (2018). Profiling human breast epithelial cells using single cell RNA sequencing identifies cell diversity. Nat Commun 9, 2028.
Nieto, M. A. (2013). Epithelial Plasticity: A Common Theme in Embryonic and Cancer Cells. Science 342.
Nieto, M. A. (2017). Context-specific roles of EMT programmes in cancer cell dissemination. Nat Cell Biol 19, 416-418.
Nieto, M. A., et al. (2016). EMT: 2016. Cell 166, 21-45.
Oliemuller, E., et al. (2017). SOX11 promotes invasive growth and ductal carcinoma in situ progression: SOX11 promotes invasion and DCIS progression. J. Pathol 243, 193-207.
Shepherd, J. H., et al. (2016). The SOX11 transcription factor is a critical regulator of basal-like breast cancer growth, invasion, and basal-like gene expression. Oncotarget 7.
Siegel, R. L., et al. (2020). Cancer statistics, 2020. CA A Cancer J Clin 70, 7-30.
Xu, Y., et al. (2017). Breast tumor cell-specific knockout of Twist1 inhibits cancer cell plasticity, dissemination, and lung metastasis in mice. Proc Natl Acad Sci USA 114, 11494-11499.
Yang, X., et al., lodine promotes thyroid cancer development via SPANXA1 through the PI3K/AKT signalling pathway. Oncol Lett. 2019 July; 18 (1): 637-644.
Ye, X., et al. (2015). Distinct EMT programs control normal mammary stem cells and tumour-initiating cells. Nature 525, 256-260.
Zhang, J., et al. (2012). SOX4 Induces Epithelial-Mesenchymal Transition and Contributes to Breast Cancer Progression. Cancer Research 72, 4597-4608. 77

1.-39. (canceled)

40. An RNA molecule that (i) specifically binds to an mRNA selected from the group consisting of SOX11 mRNA, SNAI1 mRNA, SNAI2 mRNA, ZEB1 mRNA, CCNB1 mRNA, CCNB2 mRNA, CCNA2 mRNA, CDK1 mRNA, SPANXA1 mRNA, SPANXB1 mRNA, POSTN mRNA, and MMP2 mRNA, and (ii) has a 3′ residue that is either U or A.

41. The RNA molecule of claim 40, wherein the RNA molecule's two 3′ residues are selected from the group consisting of UU, AA, AU, and UA.

42. The RNA molecule of claim 40, wherein the RNA molecule is fewer than 25 residues in length.

43. An siRNA molecule comprising (i) the RNA molecule of claim 40 as a sense strand, and (ii) an antisense RNA strand non-covalently bound thereto.

44. A cell containing the siRNA molecule of claim 40.

45. A vector comprising a nucleotide sequence that encodes at least one strand of the siRNA molecule of claim 43.

46. A cell containing the vector of claim 45.

47. A composition comprising a plurality of the siRNA molecule of claim 43, wherein (i) the composition comprises a plurality of siRNA molecule populations, and (ii) each of the siRNA molecule populations is directed to a different target mRNA than the other populations.

48. The composition of claim 47, wherein the composition comprises two siRNA molecule populations.

49. The composition of claim 47, wherein the composition comprises three siRNA molecule populations.

50. A pharmaceutical composition comprising the siRNA molecule of claim 43 and a pharmaceutically acceptable carrier.

51. A pharmaceutical composition comprising the composition of claim 47 and a pharmaceutically acceptable carrier.

52. A method for treating a subject afflicted with breast cancer comprising administering to the subject a therapeutically effective amount of the pharmaceutical composition of claim 50.

53. The method of claim 52, wherein the breast cancer is selected from the group consisting of luminal A breast cancer, luminal B breast cancer, HER2-positive breast cancer, and triple negative breast cancer.

54. The method of claim 52, wherein the breast cancer is metastatic breast cancer.

55. The method of claim 52, wherein the subject is human.


SEQUENCE LISTING (122 sequences)

SEQ ID NO: 1 - RNA, 21 nt, synthetic construct
  source 1..21: mol_type = other RNA
ggagagaattctacatttatt

SEQ ID NO: 2 - RNA, 21 nt, synthetic construct
  source 1..21: mol_type = other RNA
ggtggagttaaagtgaaattt

SEQ ID NO: 3 - RNA, 21 nt, synthetic construct
  source 1..21: mol_type = other RNA
ccagcattctcttattaattt

SEQ ID NO: 4 - RNA, 21 nt, synthetic construct
  source 1..21: mol_type = other RNA
ggagacacaagctcataattt

SEQ ID NO: 5 - RNA, 21 nt, synthetic construct
  source 1..21: mol_type = other RNA
gcccaaggaatttgagaaatt

SEQ ID NO: 6 - RNA, 21 nt, synthetic construct
  source 1..21: mol_type = other RNA
ggtggaaactttaagtaaatt

SEQ ID NO: 7 - RNA, 21 nt, synthetic construct
  source 1..21: mol_type = other RNA
gggcaaattatttgtagaatt

SEQ ID NO: 8 - RNA, 21 nt, synthetic construct
  source 1..21: mol_type = other RNA
gccgtttccagatttgaattt

SEQ ID NO: 9 - RNA, 21 nt, synthetic construct
  source 1..21: mol_type = other RNA
ggaggatgaagtgtttgattt

SEQ ID NO: 10 - RNA, 21 nt, synthetic construct
  source 1..21: mol_type = other RNA
gcctctactacagcttcaatt

SEQ ID NO: 11 - RNA, 21 nt, synthetic construct
  source 1..21: mol_type = other RNA
gccactggatagtcttcaatt

SEQ ID NO: 12 - RNA, 21 nt, synthetic construct
  source 1..21: mol_type = other RNA
gagctgttatcttagtttatt

SEQ ID NO: 13 - RNA, 27 nt, synthetic construct
  source 1..27: mol_type = other RNA
cagatgtcaagaagtaccagtgccatt

SEQ ID NO: 14 - RNA, 21 nt, synthetic construct
  source 1..21: mol_type = other RNA
gccttcaactgcaaatacttt

SEQ ID NO: 15 - RNA, 21 nt, synthetic construct
  source 1..21: mol_type = other RNA
ccgggcaatttaacaatgttt

SEQ ID NO: 16 - RNA, 21 nt, synthetic construct
  source 1..21: mol_type = other RNA
cctgggaggaagatgtttatt

SEQ ID NO: 17 - RNA, 21 nt, synthetic construct
  source 1..21: mol_type = other RNA
gggaggaagatgtttacattt

SEQ ID NO: 18 - RNA, 21 nt, synthetic construct
  source 1..21: mol_type = other RNA
gtgactaactatgcaataatt

SEQ ID NO: 19 - RNA, 21 nt, synthetic construct
  source 1..21: mol_type = other RNA
cccactcagatgtcaagaatt

SEQ ID NO: 20 - RNA, 21 nt, synthetic construct
  source 1..21: mol_type = other RNA
gggcaatttaacaatgtcttt

SEQ ID NO: 21 - RNA, 21 nt, synthetic construct
  source 1..21: mol_type = other RNA
gcagactagagtctgagattt

SEQ ID NO: 22 - RNA, 21 nt, synthetic construct
  source 1..21: mol_type = other RNA
caggactctaatccagagttt

SEQ ID NO: 23 - RNA, 21 nt, synthetic construct
  source 1..21: mol_type = other RNA
cacaggagaatgtattaaatt

SEQ ID NO: 24 - RNA, 21 nt, synthetic construct
  source 1..21: mol_type = other RNA
gacccacacattaccttgttt

SEQ ID NO: 25 - RNA, 21 nt, synthetic construct
  source 1..21: mol_type = other RNA
gtgctgaccaaccaaataatt

SEQ ID NO: 26 - RNA, 21 nt, synthetic construct
  source 1..21: mol_type = other RNA
gctagattgagagaataaatt

SEQ ID NO: 27 - RNA, 21 nt, synthetic construct
  source 1..21: mol_type = other RNA
gaagccaaatgacaaataatt

SEQ ID NO: 28 - RNA, 21 nt, synthetic construct
  source 1..21: mol_type = other RNA
gacgcaatcaatgtttacttt

SEQ ID NO: 29 - RNA, 21 nt, synthetic construct
  source 1..21: mol_type = other RNA
gcacaaacatgaggaatcttt

SEQ ID NO: 30 - RNA, 21 nt, synthetic construct
  source 1..21: mol_type = other RNA
gggaaagattagctttgaatt

SEQ ID NO: 31 - RNA, 21 nt, synthetic construct
  source 1..21: mol_type = other RNA
gaggattcttacaaattcatt

SEQ ID NO: 32 - RNA, 21 nt, synthetic construct
  source 1..21: mol_type = other RNA
gacacacatacagtgattatt

SEQ ID NO: 33 - RNA, 21 nt, synthetic construct
  source 1..21: mol_type = other RNA
ccactgtacaaagaattgttt

SEQ ID NO: 34 - RNA, 21 nt, synthetic construct
  source 1..21: mol_type = other RNA
ggcgcaataacgttacaaatt

SEQ ID NO: 35 - RNA, 21 nt, synthetic construct
  source 1..21: mol_type = other RNA
ggcaagtgttggagaataatt

SEQ ID NO: 36 - RNA, 21 nt, synthetic construct
  source 1..21: mol_type = other RNA
gccctatcaatgtgacaaatt

SEQ ID NO: 37 - RNA, 21 nt, synthetic construct
  source 1..21: mol_type = other RNA
ggcggtagatggtaatgtatt

SEQ ID NO: 38 - RNA, 21 nt, synthetic construct
  source 1..21: mol_type = other RNA
ggaggaagaagtggaagaatt

SEQ ID NO: 39 - RNA, 21 nt, synthetic construct
  source 1..21: mol_type = other RNA
gaaccaggcaaagtaaatatt

SEQ ID NO: 40 - RNA, 21 nt, synthetic construct
  source 1..21: mol_type = other RNA
gatccagccaaatggaaattt

SEQ ID NO: 41 - RNA, 21 nt, synthetic construct
  source 1..21: mol_type = other RNA
ctcagaaccagttgtaaattt

SEQ ID NO: 42 - RNA, 21 nt, synthetic construct
  source 1..21: mol_type = other RNA
ctctgaaagaacacattaatt

SEQ ID NO: 43 - RNA, 21 nt, synthetic construct
  source 1..21: mol_type = other RNA
gagcaagtgtctgaagaaatt

SEQ ID NO: 44 - RNA, 21 nt, synthetic construct
  source 1..21: mol_type = other RNA
gtctgggtgtaatcgtaaatt

SEQ ID NO: 45 - RNA, 21 nt, synthetic construct
  source 1..21: mol_type = other RNA
ggctagtacaggttcaaattt

SEQ ID NO: 46 - RNA, 21 nt, synthetic construct
  source 1..21: mol_type = other RNA
gagcctgttaaagaagaaatt

SEQ ID NO: 47 - RNA, 21 nt, synthetic construct
  source 1..21: mol_type = other RNA
gtgactgacaacacttatatt

SEQ ID NO: 48 - RNA, 21 nt, synthetic construct
  source 1..21: mol_type = other RNA
ctggctagtacaggttcaatt

SEQ ID NO: 49 - RNA, 21 nt, synthetic construct
  source 1..21: mol_type = other RNA
caggctttctctgatgtaatt

SEQ ID NO: 50 - RNA, 21 nt, synthetic construct
  source 1..21: mol_type = other RNA
gctgatccaaacctttgtatt

SEQ ID NO: 51 - RNA, 21 nt, synthetic construct
  source 1..21: mol_type = other RNA
ggactgtcaagaacaagtatt

SEQ ID NO: 52 - RNA, 21 nt, synthetic construct
  source 1..21: mol_type = other RNA
gacagatggaaatgaagattt

SEQ ID NO: 53 - RNA, 21 nt, synthetic construct
  source 1..21: mol_type = other RNA
gatcggttcatgcagaatatt

SEQ ID NO: 54 - RNA, 21 nt, synthetic construct
  source 1..21: mol_type = other RNA
gcactttcctccttctcaatt

SEQ ID NO: 55 - RNA, 21 nt, synthetic construct
  source 1..21: mol_type = other RNA
cactctaccacagctgaattt

SEQ ID NO: 56 - RNA, 21 nt, synthetic construct
  source 1..21: mol_type = other RNA
ggccaagaatgtggtgaaatt

SEQ ID NO: 57 - RNA, 21 nt, synthetic construct
  source 1..21: mol_type = other RNA
gcagcaaactcctgaagattt

SEQ ID NO: 58 - RNA, 21 nt, synthetic construct
  source 1..21: mol_type = other RNA
ccagtgatttggagaatattt

SEQ ID NO: 59 - RNA, 21 nt, synthetic construct
  source 1..21: mol_type = other RNA
ggtgcattatcatccttcttt

SEQ ID NO: 60 - RNA, 21 nt, synthetic construct
  source 1..21: mol_type = other RNA
gttcccaaatccgagaaattt

SEQ ID NO: 61 - RNA, 21 nt, synthetic construct
  source 1..21: mol_type = other RNA
ggacattgataacgaagattt

SEQ ID NO: 62 - RNA, 21 nt, synthetic construct
  source 1..21: mol_type = other RNA
gctcttggcttccaagtattt

SEQ ID NO: 63 - RNA, 21 nt, synthetic construct
  source 1..21: mol_type = other RNA
gcacactttagccaagtattt

SEQ ID NO: 64 - RNA, 21 nt, synthetic construct
  source 1..21: mol_type = other RNA
gtgactacgttaaggatattt

SEQ ID NO: 65 - RNA, 21 nt, synthetic construct
  source 1..21: mol_type = other RNA
gtcctctggtctatctcattt

SEQ ID NO: 66 - RNA, 21 nt, synthetic construct
  source 1..21: mol_type = other RNA
gctggcctgaatcattaattt

SEQ ID NO: 67 - RNA, 21 nt, synthetic construct
  source 1..21: mol_type = other RNA
gcctcaaagtttgaagaaatt

SEQ ID NO: 68 - RNA, 21 nt, synthetic construct
  source 1..21: mol_type = other RNA
gtggccaagtacaaattattt

SEQ ID NO: 69 - RNA, 21 nt, synthetic construct
  source 1..21: mol_type = other RNA
gagggtgggaagcttatattt

SEQ ID NO: 70 - RNA, 21 nt, synthetic construct
  source 1..21: mol_type = other RNA
ccagctgtcaggataataatt

SEQ ID NO: 71 - RNA, 21 nt, synthetic construct
  source 1..21: mol_type = other RNA
gtggcaagatgagagtaaatt

SEQ ID NO: 72 - RNA, 21 nt, synthetic construct
  source 1..21: mol_type = other RNA
gccagtgagtgttaatgaatt

SEQ ID NO: 73 - RNA, 21 nt, synthetic construct
  source 1..21: mol_type = other RNA
gaccctcaaattctgacattt

SEQ ID NO: 74 - RNA, 21 nt, synthetic construct
  source 1..21: mol_type = other RNA
ggatcttcctgtaaatgattt

SEQ ID NO: 75 - RNA, 21 nt, synthetic construct
  source 1..21: mol_type = other RNA
gcatatctgaatacagtattt

SEQ ID NO: 76 - RNA, 21 nt, synthetic construct
  source 1..21: mol_type = other RNA
cgggaaatttctctattaatt

SEQ ID NO: 77 - RNA, 21 nt, synthetic construct
  source 1..21: mol_type = other RNA
caggactataagaatacattt

SEQ ID NO: 78 - RNA, 21 nt, synthetic construct
  source 1..21: mol_type = other RNA
cctggctaaagatgaatattt

SEQ ID NO: 79 - RNA, 21 nt, synthetic construct
  source 1..21: mol_type = other RNA
ggcactgaatcatccatattt

SEQ ID NO: 80 - RNA, 21 nt, synthetic construct
  source 1..21: mol_type = other RNA
gccttggtcagagtaataatt

SEQ ID NO: 81 - RNA, 21 nt, synthetic construct
  source 1..21: mol_type = other RNA
gtctcactgtaacaactattt

SEQ ID NO: 82 - RNA, 21 nt, synthetic construct
  source 1..21: mol_type = other RNA
gcaccatatttgctgaacttt

SEQ ID NO: 83 - RNA, 21 nt, synthetic construct
  source 1..21: mol_type = other RNA
gatgtagctttctgacaaatt

SEQ ID NO: 84 - RNA, 21 nt, synthetic construct
  source 1..21: mol_type = other RNA
gttcttcacagagacttaatt

SEQ ID NO: 85 - RNA, 21 nt, synthetic construct
  source 1..21: mol_type = other RNA
cagctgtacttcgtcttcttt

SEQ ID NO: 86 - RNA, 21 nt, synthetic construct
  source 1..21: mol_type = other RNA
gaggaattcatggaaataatt

SEQ ID NO: 87 - RNA, 21 nt, synthetic construct
  source 1..21: mol_type = other RNA
ctgccactgacattgaagatt

SEQ ID NO: 88 - RNA, 21 nt, synthetic construct
  source 1..21: mol_type = other RNA
cccgagagaacagaatcaatt

SEQ ID NO: 89 - RNA, 21 nt, synthetic construct
  source 1..21: mol_type = other RNA
cgctacaggaggaactttatt

SEQ ID NO: 90 - RNA, 21 nt, synthetic construct
  source 1..21: mol_type = other RNA
gtagcaagaagctacatcttt

SEQ ID NO: 91 - RNA, 21 nt, synthetic construct
  source 1..21: mol_type = other RNA
catggaaataatggttgaatt

SEQ ID NO: 92 - RNA, 21 nt, synthetic construct
  source 1..21: mol_type = other RNA
cagaggaactgctgaatgatt

SEQ ID NO: 93 - RNA, 21 nt, synthetic construct
  source 1..21: mol_type = other RNA
ctgacattgaagaaccaattt

SEQ ID NO: 94 - RNA, 21 nt, synthetic construct
  source 1..21: mol_type = other RNA
ccatactagtggttcgctatt

SEQ ID NO: 95 - RNA, 21 nt, synthetic construct
  source 1..21: mol_type = other RNA
ccaatatatacaatggacatt

SEQ ID NO: 96 - RNA, 21 nt, synthetic construct
  source 1..21: mol_type = other RNA
gccactgcgaagattcaaatt

SEQ ID NO: 97 - RNA, 21 nt, synthetic construct
  source 1..21: mol_type = other RNA
ggaggaggaattcatagaatt

SEQ ID NO: 98 - RNA, 21 nt, synthetic construct
  source 1..21: mol_type = other RNA
catcgaagaaccaatatattt

SEQ ID NO: 99 - RNA, 21 nt, synthetic construct
  source 1..21: mol_type = other RNA
cctcaaacttcggcaatgatt

SEQ ID NO: 100 - RNA, 21 nt, synthetic construct
  source 1..21: mol_type = other RNA
cgaagaaccaatatatacatt

SEQ ID NO: 101 - RNA, 21 nt, synthetic construct
  source 1..21: mol_type = other RNA
catagaaataacgactgaatt

SEQ ID NO: 102 - RNA, 21 nt, synthetic construct
  source 1..21: mol_type = other RNA
caagaagctacatccctcatt

SEQ ID NO: 103 - RNA, 21 nt, synthetic construct
  source 1..21: mol_type = other RNA
gctggcacctgtgaataattt

SEQ ID NO: 104 - RNA, 21 nt, synthetic construct
  source 1..21: mol_type = other RNA
cctggagactggacattattt

SEQ ID NO: 105 - RNA, 21 nt, synthetic construct
  source 1..21: mol_type = other RNA
gccctggttatatgagaattt

SEQ ID NO: 106 - RNA, 21 nt, synthetic construct
  source 1..21: mol_type = other RNA
ggtcctaattcctgattcttt

SEQ ID NO: 107 - RNA, 21 nt, synthetic construct
  source 1..21: mol_type = other RNA
gccatcacatcggacatattt

SEQ ID NO: 108 - RNA, 21 nt, synthetic construct
  source 1..21: mol_type = other RNA
ggatctagaagacgattaatt

SEQ ID NO: 109 - RNA, 21 nt, synthetic construct
  source 1..21: mol_type = other RNA
gagccttgtatgtatgttatt

SEQ ID NO: 110 - RNA, 21 nt, synthetic construct
  source 1..21: mol_type = other RNA
ctgctggaaatacttaatatt

SEQ ID NO: 111 - RNA, 21 nt, synthetic construct
  source 1..21: mol_type = other RNA
gtagcaccttcaaagaaattt

SEQ ID NO: 112 - RNA, 21 nt, synthetic construct
  source 1..21: mol_type = other RNA
ggcatgattattccttcaatt

SEQ ID NO: 113 - RNA, 21 nt, synthetic construct
  source 1..21: mol_type = other RNA
gccctgaagaatcagcaattt

SEQ ID NO: 114 - RNA, 21 nt, synthetic construct
  source 1..21: mol_type = other RNA
gtggccaactacaacttcttt

SEQ ID NO: 115 - RNA, 21 nt, synthetic construct
  source 1..21: mol_type = other RNA
gaggtttgcttcctctttatt

SEQ ID NO: 116 - RNA, 21 nt, synthetic construct
  source 1..21: mol_type = other RNA
ccaccacctacaactttgatt

SEQ ID NO: 117 - RNA, 21 nt, synthetic construct
  source 1..21: mol_type = other RNA
ggtcagagcttccaaagtatt

SEQ ID NO: 118 - RNA, 21 nt, synthetic construct
  source 1..21: mol_type = other RNA
gcacccatttacacctacatt

SEQ ID NO: 119 - RNA, 21 nt, synthetic construct
  source 1..21: mol_type = other RNA
gaggcagacatcatgatcatt

SEQ ID NO: 120 - RNA, 21 nt, synthetic construct
  source 1..21: mol_type = other RNA
ctcactcctacctggtaattt

SEQ ID NO: 121 - RNA, 21 nt, synthetic construct
  source 1..21: mol_type = other RNA
gtgcctattacctgaagcttt

SEQ ID NO: 122 - RNA, 21 nt, synthetic construct
  source 1..21: mol_type = other RNA
gtgcccaagaatagatgcttt